(19) United States
(12) Patent Application Publication
(10) Pub. No.: US 2004/0047927 A1
(43) Pub. Date: Mar. 11, 2004

(54) Soybean drug and new method of extracting soybean isoflavone

(76) Inventor: Yaguang Liu, Flushing, NY (US)

Correspondence Address: Yaguang Liu, 67-08 168th Street, Flushing, NY, 11365, US

(21) Appl. No.: 10/234,477

(22) Filed: Sep. 5, 2002

(51) Int. Cl. A61K 35/78

(52) U.S. Cl. 424/757

(57) ABSTRACT

This invention relates to a new method of isolating soybean isoflavone (SIS) from soybean residue and leaves Soybean isoflavone can treat and prevent cardiovascular disease, menopausal and climacteric syndrome and increase immune function

### BACKGROUND OF THE INVENTION

This invention relates to a new method of isolating soybean isoflavone (SIS) from soybean residue and leaves Soybean isoflavone can treat and prevent cardiovascular disease, menopausal and climacteric syndrome, and increase immune function.

### DESCRIPTION OF THE PRIOR ART

Recent several articles reported that soy protein has important action in decreases serum cholesterols For example, James W. Anderson, et al reported that ingestion of soy protein can decrease total cholesterol and low-density lipoprotein (LDL) cholesterol when soy protein intake averaged 47 g per day but the ingestion of soy protein cannot increase in serum concentrations of high-density lipoprotein (HDL) cholesterol”. (James W Anderson et al: The New England Journal of Medicine 333:276-282, 1995).

The ingredients of soybean including isoflavones and saponins may be useful in treatment and prevention of cardiovascular disease and climacteric melancholia or climacteric syndrome The traditional method of isolating soybean isoflavone is extracting isoflavone from whole soybean and it needs to use many organic solvents The cost of above process is more expensive Meanwhile, organic solvents are not good for health of human. After soybean isoflavone is extracted from soybean, all residual materials of soybean are a waste Additional, the content of isoflavone in soybean is very low, lower than 0 01%. Therefore, the traditional method of extracting of isoflavone from whole soybean makes a huge waste which equals about 99.99% of soybean weight Also, leaves of soybean are waste too. Therefore, extracting isolavone from soybean residue and leaves has a great economical and environmental value.

### DETAILED DESCRIPTION OF THE INVENTION

The soybean isoflavone, according to this invention can be obtained by new extracting from soybean residue and leaves SIS can treat and prevent cardiovascular disease, menopausal and climacteric syndrome, and increase immune function.

As James W Anderson reported that soy protein can decrease serum lipids but he indicated, unfortunately, that soy protein needs intake 47 g average per day 47 g soy protein per day is very big amount for consumption For example, 47 g soy protein equals 12 boxes (each box has 10 25 oz, 290 g net weight) of Tofu Therefore, if a person needs intake average 47 g soy protein per day, he or she almost needs to eat a lot of soy-containing food every day It is very inconvenience Also, many people cannot eat soy containing food only but don't eat traditional food

This invention relates to isoflavone of soybean for increasing immune function, treating cardiovascular disease and decreasing serum lipids. It is important that the dosage of SIS is about 20 mg per day. One time Person one time just takes one capsule It is very convenience. The dosage of SIS is less than 0 1% of dosage of soy protein only. Also, SIS has important functions including decreasing serum lipids, increasing immune function and treating cardiovascular disease But soy protein is usefully decreasing lipids only.

Meanwhile, the dosage of isoflavone of soybean is small. Therefore, SIS can be used as drug, health food or food additives, which can be added into other food It is a very convenient way too.

So far, the main purpose of uses of soybean is used for extracting soybean oil. Soybean oil provides 58 9% of visible fats (animal fats provides 16 1%, palm and coconut oils provide 15.3% and other vegetable oil 9 7%) Since 40 to 50% of total calories in the American diet are contributed by fats, the dietary and nutritional importance of soybean oil are great Soybean oil is high in polyunsaturated fatty acid and has a high vitamin E content Current usage of soybean in food is at level of some 4 5 billion kg per year which represents over 80% of all vegetable oils and oil products currently consumed in foods. Every single day, Americans consume roughly 11 million kilos of soybean oil. And soybean oil provides almost 7.3 billion pounds of visible fat The weight percentage of soybean oil is about 13-24% of whole soybean, and it depends different genus of soybean Therefore, about 40 billion pounds of soybean residue are produced after extracting soybean oil by the factories. So far, soybean residue is almost a waste and soybean leaves are totally a waste.

During climacteric age, women have the following signs and symptoms, such as level of estrogen and progesterone drop in the body, pituitary changed, loss of bone density, hot flashes, felling of intense heat, genital bleeding, some women may lead to vertebral, hip and wrist fractures, lipid level becomes higher and higher lipid level will cause cardiovascular disease. However, if using estrogens for treating climacteric syndrome, it will cause many serious side effects, which included carcinogenesis. But, SIS can be safely used for treatment and prevention of cardiovascular disease and climacteric syndrome SIS also has no side effects.

This invention relates to a new extracting method of producing SIS from soybean residue and leaves Soybean residue is by-product after extracting oil Soybean leaves are a waster.

As mentioned above, the amount of soybean residue and leaves are very huge and they are a waste. Obviously, extracting SIS from soybean's residue and leaves has great economic and environmental value.

Additional, it is important that the process of extracting oil from soybean includes many steps of extracting of organic solvents which can move fats, pigment, sugar and other impurities into organic solvents and water from soybean Other words, soybean residue does almost not contain fats, pigment sugar of soybean In fact, soybean residue is more proper for extracting isoflavone than whole soybean For the reason given above, we can see that the process of extracting SIS from soybean residue is more simple and easy than the process of extracting SIS from whole soybean. Extracting isoflavone from soybean residue can save a lot of organic solvents. Therefore, the cost of this new extracting SIS from soybean residue and leaves is cheaper than the process of extracting SIS from whole soybean

It is, therefore, a primary object of the present invention to provide a new method of extracting SIS from soybean residue and leaves, which is a waste.

This new method has great economic and environmental value It is a further related object to provide SIS use for treating and preventing cardiovascular disease, menopausal and climacteric syndrome, and increasing immune function

The following specific examples will provide detailed illustrations of methods of producing SIS according to the present invention and pharmaceutical dosage units containing SIS. Moreover, examples will be given of pharmaceutical testing performed with SIS, which demonstrates its effectiveness in treating and preventing cardiovascular disease and menopausal and climacteric syndrome These examples are not intended, however, to limit or restrict the scope of the invention in any way, and should not be construed as providing conditions, parameters, reagents, or starting materials which must be utilized exclusively in order to practice the present invention.

### EXAMPLE 1

### Extraction of SIS from Soybean Residue

After soybean was extracted oil, residue of soybean was solid which named soybean residue (CR) CR was obtained from factory of manufacture soybean oil or purchased from market 5L of 95% ethanol was added to 1 kg of ground powder of CR and allowed to stand to one day at room temperature. The solution was filtered and extracting filtrate saved.

2L of ethanol was added to the filtered residue and refluxed in water both for 2 hours The refluxing was repeated twice by collecting the ethanol, replacing it with an equal volume of fresh 95% ethanol and refluxing for 2 hours. The refluxed ethanol was cooled and filtered and the filtrate combined with the extract filtrate. Ethanol was then recovered by reduced pressure distillation and the residue dissolved in 3L of acetone Acetone was then recovered by reduced pressure distillation The distillated residue was suspended in water Then water mixture solution was extracted by acetic ether Acetic ether was then recovered by reduced pressure distillation and the distillated residue was dried under vacuum The resulting white powder was product i e soybean isoflavone Products were identified by ultraviolet infrared, mass NMR spectra.

### EXAMPLE 2

### Extraction of SIS from Soybean Leaves

Soybean leaves are obtained from farms. 5L of 95% ethanol was added to 1 kg of ground powder of soybean leaves and allowed to stand to one day at room temperature The solution was filtered and extracting filtrate saved.

2,000 ml of ethanol was added to the filtered residue and refluxed in water both for 2 hours The refluxing was repeated twice by collecting the ethanol, replacing it with an equal volume of fresh 95% ethanol and refluxing for 6 hours The refluxed ethanol was cooled and filtered and the filtrate combined with the extract filtrate. Ethanol was then recovered by reduced pressure distillation and the residue was suspended in water and then water mixture was extracted by chloroform. The chloroform was recovered by reduced pressure to yield chloroform extract. The chloroform extract was extracted by butanol The extract of butanol was purified in column packed with sephadex column and eluting with methanol Elution was extracted by acetone. Acetone was then recovered by reduced pressure distillation The distillated residue was extracted by acetic ether. Then acetic ether was then recovered by reduced pressure distillation and the distillated residue was dried under vacuum. The resulting white powder was product i.e. soybean isoflavone. Products were identified by ultraviolet, infrared, mass, NMR spectra.

The following examples are related to pharmacological tests

### EXAMPLE 3

### Effect of SIS on Human Coronary Artery Endothelial Cells (HCAEC) and Oxidative Modified Low Density Lipoprotein (ox-LDL)

Low density lipoprotein and its oxidative modified form (ox-LDL) is critical risk factor in cardiovascular disease, especially in atherogenesis LDL is oxidized in HCAEC and produced (ox-LDL) a very highly injurious product which caused cells dysfunction in arteries It is known that ox-LDL appears to induce dysfunction of HCAEC, and endothelial uptake of ox-LDL is a key factor in atherosclerosis. Therefore, using drug for decreasing uptake of ox-LDL by HCAEC cell is very important. So far, no this kind of drugs can decrease uptake of ox-LDL by HCAEC and it also has no side effects

Methods

HCAEC growth medium consisted of endothelial cell basal medium. Growth medium consisted of 500 ml of endothelial cell basal medium, 5 ng of human epidermal growth factor, 5 mg of hydrocortisone, 25 mg of gentamicin and 25 μg of amphotericin B, and 25 ml fetal bovine serum HCAECs were seeded in a 25 cm² flask (4,000 cells/cm²), incubated at 37° C. in 95% air-5% CO₂, Fifth generation HCAECs (1×10⁶) were used to experiment. [I¹²⁵]-ox-LDL was prepared and [I¹²⁵]-ox-LDL were kept in 50 mM Tris-HCl, 0.15 M NaCl and 2 mM EDTA at pH 7.4, and were used within 10 days of preparation. HCAECs incubated with drugs and [I¹²⁵]-ox-LDL (50 μg/ml) Different groups of HCAECs were dissolved in sample buffer Cells were homogenized and [I¹²⁵]-ox-LDL was determined.

Results

The experimental results are listed in the following table.

TABLE 1

| Group | Percentage of uptake ox-LDL (%) |
|---|---|
| Control | 100 |
| SIS | |
| 10 μg/ml | 72.3 ± 8.0 |
| 50 μg/ml | 60.8 ± 7.1 |
| 100 μg/ml | 40.9 ± 5.6 |

The data of Table I indicated that SIS could obviously decrease uptake ox-LDL by HCAEC It means that SIS can treat and prevent atherosclerosis

### EXAMPLE 4

### Effect of SIS on Aortic Smooth Muscle Cells

The atherosclerosis caused by high level of LDL and aggregation of platelet (thrombocyte) which caused plaques Smooth muscle cells (SMC) were found in both plaques and fatty streaks in atherosclerosis. Proliferation of SMC was a key factor for inducing blood plaques and then became atherosclerosis Therefore, drug, which inhibited growth of SMC, has an important value.

Method

The aortic smooth muscle cells were cultured in DMEM with penicillin, streptomycin (100 mg/L), and NaHCO₂, (24 mmol/L) containing 10% FBS, plated in tissue-culture flasks and incubated under standard tissue culture conditions. SMC were plated at a density of 1×10⁶ cells/well in 24-well tissue-culture dishes and allowed to grow to subconfluence in DMEM containing 10% FBS under standard tissue culture conditions After three cell washes with PBS, the growth of SMC was arrested by adding DMEM containing 0 4% FBA for 48 hours SMC were treated with a concentration of 0 (control) or 10, 50, 100 μg/m SIS in fresh DMEM containing 10% FBS and allowed to grow for 4 days Then SBC were cultured with ³H-TdR under standard tissue culture conditions for determination of DNA synthesis.

Results

The experimental results are listed in the following table.

TABLE 2

| | DNA synthesis |
|---|---|
| | ³H-TdR CPM |
| Control | 2340 ± 360 |
| SIS | |
| 10 μg/ml | 1681 ± 201 |
| 50 μg/ml | 1248 ± 128 |
| 100 μg/ml | 969 ± 121 |

The above data of Table 2 indicated that SIS strongly inhibited DNA synthesis of SMC It means SIS can treat and prevent atherosclerosis.

### EXAMPLE 5

### The Effect of SIS on Myocardial Nutritious Blood Flow in Mice

In the present example the effect of the myocardial uptake of ⁸⁶Rb (Rubidium) used as the index of myocardial nutritious blood flow. The male mice weight 18-22 g were used in the experiments and were divided into treated (SIS) and control group. The dosage of SIS was 100 mg/kg injected intraperitoneally. The control mice were injected with same volume of normal saline. These injections were repeated daily for four days On the last day, ⁸⁶Rb 50 μc/kg body weight was administered by tail vein for both SIS and control group, the injections to be completed within 3 seconds for every mouse 30 seconds after administration of ⁸⁶Rb the heart was excised. The heart was then dissected and weighted after the removal of auricles and blotted with filter paper quickly The heart was digested by 1 25 N NaOH in a boiled water bath. Then dry digested solution with drier. The ⁸⁶Rb uptakes were determined on a scintillator. The coronary blood flow was expressed as per minute per gram heart weight in pulse to amount of ⁸⁶Rb given (CPM/g)

The experimental results are listed in the following table.

TABLE 3

| | Control | | SIS |
|---|---|---|---|
| CPM/g | 120 ± 10 | | 175 ± 16.0 |
| Number of samples | 20 | | 20 |
| P | | <0.01 | |

The data of Table 3 indicated that SIS could obviously increase myocardial blood flow.

### EXAMPLE 6

### The Effect of SIS on Coronary Flow

The male rats (280 to 320 g body weight), maintained on a standard diet, were used in these experiments. The rats were lightly anaesthetized with diethyl ether The left femoral vein was exposed and heparin (200 IU) was administered intravenously. One minute (min) after administration of heparin, the heart was excised and placed in 4° C. perfusion medium until contraction had ceased The heart was then mounted on the perfusion apparatus. Bicarbonate (PH 7.4) buffer was the standard perfusion fluid. The perfusion fluid was maintained at 37° C. and in aerobic studies, the fluid was equilibrated with air +CO₂ (95.5). The heart was perfused after mounting immediately for a 5 min wash-out period The preparation was then converted into working heart system for a 15 min period (standard perfusion medium plus 11 mM glucose) Flow meter calibrate for flow (5 to 670 ml/min) at 37° C. was used to measure aortic flow rates SIS was included separately in perfusion medium throughout the experimental time course

Statistical comparison between control and SIS group was made by Student's T-test with the significance level being p<0.05. The values given are means+standard error (S.E.M.).

The experimental results are listed in the following table.

TABLE 4

| | | Coronary | |
|---|---|---|---|
| Group | | flow (ml/min) | Number of samples |
| After treatment of SIS | 0 | 6.7 ± 0.6 | 10 |
| (minute) | 1′ | 9.8 ± 0.8 | 10 |
| | 3′ | 10.2 ± 0.8 | 10 |
| | 5′ | 9.9 ± 0.9 | 10 |
| | 10′ | 8.0 ± 0.5 | 10 |
| | 15′ | 7.8 ± 0.4 | 10 |
| | 20′ | 7.8 ± 0.5 | 10 |

The data of Table 4 indicated that SIS could obviously increase coronary flow

### EXAMPLE 7

### The Effect of SIS on the Survival Percentage of Mice Under Hypoxia

The male mice of weight 18-20 g were used in the experiments and were divided into SIS and control group. The dosage of SIS was 50 mg/kg injected intraperitoneally. The control mice were injected with same volume of normal saline These injections were repeated daily for four days On the last day, both SIS and control group mice have been placed in airtight box Atmospheric pressure of airtight box was reduced to 180 mm Hg with air pump. SIS group resulted in a prolongation of survival time and elevation of survival percentage of the mice under hypoxia

The experimental results are listed in the following table

TABLE 5

| | Survival time (min) | Survival percentage (%) |
|---|---|---|
| Control | 16 ± 2 | 0 |
| SIS | 48 ± 6 | 56.8 |
| P | <0.1 | <0.01 |

The data of Table 5 indicated that SIS might improve the oxygen utilization during hypoxia.

Above data of Table 3-5 indicated that SIS could treat cardiovascular disease

### EXAMPLE 8

### The Effect of SIS on Serum Lipids

The male mice of weight 18-20 g were used in the experiments and were divided into SIS and control group The dosage of SIS was 50 mg/kg injected intraperitoneally The control mice were injected with same volume of normal saline. These injections were repeated daily for four days Control group. oral administration of a high fat diet SIS group. high fat diet+SIS Normal group not administrate a high fat diet Total cholesterol (TC), triglyceride (TG) and free fatty acid (FFA) were determined.

TABLE 6

| Group | TC (mg/dl) | TG (mg/dl) | FFA (mEp/l) |
|---|---|---|---|
| Normal | 70 ± 4 | 50 ± 2 | 0.20 ± 0.01 |
| Control | 145 ± 10 | 85 ± 4 | 0.41 ± 0.02 |
| SIS | 95 ± 6 | 53 ± 3 | 0.21 ± 0.01 |
| P | <0.01* | <0.01* | <0.01* |

*Significance of difference between control group and SIS group.

The data of Table 6 indicated that SIS could significantly decrease total cholesterol, triglyceride and free fatty acid

### EXAMPLE 9

### The Effect of SIS on Immune Function

I. Animal Section

1. Inject 2 ml of normal saline into the peritoneal cavity of mouse for control group and 50 g/kg SIS for SIS group daily.

2. Kill the animal after 3 days.

3. Inject 2-5 ml of tissue culture medium into the peritoneal cavity and gently press the abdomen to bring the cells into suspension.

4. Open the abdominal skin of the mouse and hold up the center of the peritoneum with forceps.

5. Make a small hole in the peritoneum and remove the medium with a pipe

6. Finally open the mouse fully and suck out all the mediums.

7. Estimate the number of phagocytes by the uptake of a 1% neutral red solution (haemocytometer count)

II. Stained Method

Add 0.02 ml of 5% washed chick red blood cell suspension to 0.5 ml of the peritoneal exudate, shake gently to mix and incubate at 37 C for 5 minutes. Dip two coverslips, close to each other, the above mixture and incubate for 30 minutes for the migration of the macrophages along the cover slips, fix and stain with sharma stain. Examine microscopically for: Phagocytic rate−number of macrophages with phagocytized chick red blood cells per 100 macrophages counted.

Results

The results are illustrated the following table

TABLE 7

| | Normal | | SIS |
|---|---|---|---|
| Phagocytic percent ± SD (%) | 35.10 ± 2.01 | | 51.8 ± 6.7 |
| Number of samples | 12 | | 12 |
| P | | <0.01 | |

III. ⁵¹Cr labeling method:

Method—Counted the number of macrophages in the peritoneal exudate of mice and adjusted to 1×10⁶ cell/ml with normal saline. Added 0.1 ml of the macrophage suspension i e. 1×10⁶ cells to each well on the plastic plate for the rest. Labelled the chick red blood cell with ⁵¹Cr, suspend the label chick red blood cell and adjusted to 1.5×10⁶/ml, added 0 1 ml, i e. 1.5×10⁵, to each well Incubated at 37° C. for 30 minutes, washed to remove the free chick red blood cells. Counted each well in a counter.

The results are listed below table.

TABLE 8

| | Normal | | SIS |
|---|---|---|---|
| CPM | 1089 ± 341 | | 2830 ± 367 |
| Number of samples | 12 | | 12 |
| P | | <0.001 | |

The above data of Table 7 and 8 of phagocytosis test indicated that SIS could increase immune function in mice

### EXAMPLE 10

### Effect of SIS on DNA Synthesis of Uterine

Female Sprague-Dawley rats at 20 days of age, allowed free access to food and water and, as immature rats, used by age 23 days. Castrate rats were the same strain. Rats of normal group were administered in saline solution by intraperitoneal injection. Rats of control group were injected cycloheximide Rats of treatment group injected cycloheximide+SIS.

At the indicated times the rats were sacrificed by decapitation. The entire uterus from oviduct to cervix was removed, quickly dissected free of fat and connective tissue, blotted on hard filter paper, and weighed. Each uterus was minced with a scissors into a 12-ml polycarbonate tube and homogenized, 10 sec with ice cooling in 10 mM Tris-HCl, pH 7 2, buffer (10 mg of tissue per ml unless otherwise stated), with homogenizer Aliquets used for DNA analysis. Determination of DNA is used methods of ³H-TdR. The results are shown as following table

TABLE 9

| | DNA synthesis |
|---|---|
| Group | (CPM/10 g uterine tissues) |
| Normal (N) | 1250 ± 140 |
| Control (C) | 721 ± 95 |
| SIS | 1125 ± 120 |

The data of Table 9 indicated that SIS could increase DNA synthesis of uterine, which injured by cycloheximide.

### Example 11

### Effect of SIS on Receptor

Methods of animal and uterus preparation are as described in above examples Receptor preparation is described as follows.

Plasma membrane isolation from uterine tissues for the determination of receptor was carried out as the following. Tissues were excised, suspended in 10 vols (w/v) of a buffer containing 250 mM sucrose, 25 mM Tris-HCl, 1 mM EGTA (pH 7.4), and homogenized at 4° C. The crude homogenate was centrifuged to isolate the plasma membrane fraction, which was resuspended in Tris buffer and stored under liquid nitrogen for receptor analysis Ten nm [³H]estradiol was used for measuring the estrogen (ER) and progesterone (PR) binding respectively. Specific binding was determined by subtracting the non-specific binding (radiolabeled substrate+10 μl of a 200-fold molar excess of unlabeled hormone) from total binding Receptor levels were determined

Binding kinetics was determined by Scatchard analysis on all treatment groups Briefly, 50 μg of pooled uterine plasma membrane preparations were incubated with 1000, 100, 1, 0.1 and 0.01 pM radiolabeled hormone with and without 200-fold excess radioinert hormone Data were plotted as Bound/Free vs f mol hormone bound and K_{D} values were determined from the slopes of these plots.

TABLE 10

Uterine receptor levels

| Group | Estradiol | Progesterone |
|---|---|---|
| Normal (N) | 89.0 ± 13.5 | 488 ± 59 |
| Control (C) | 61.8 ± 5.5 | 250 ± 37 |
| SIS | 88.5 ± 12.0 | 420 ± 51* |

*P<0.01 compared with control group

Above data indicated that SIS increased receptors levels of estradiol and progesterone significantly

### EXAMPLE 12

### Effect of SIS on Uterine RNA

Uterine samples (100 mg) were digested in a 4 M guanidium thiocyanate solution, to which was added 50 μl of 2 M sodium acetate (pH 4 0), 500 μl phenol, and finally 200 μl chloroform/isoamyl alcohol (24 1, v/v) sequentially, with mixing after each addition. The RNA from the resultant solution was pelleted by mixing with 1 vol. isopropanol and incubating at −20° C. for 2 h and spun at 12000×g for 12 min, then resuspended in 0.5% sodium dodecyl sulfate (SDS). The RNA suspension was incubated for 10 min at 65° C. and stored at −20° C. until use RNA concentration was determined by absorbance intensity at 260 nm.

TABLE 11

| Group | RNA units |
|---|---|
| Normal (N) | 0.89 ± 0.09 |
| Control (C) | 0.50 ± 0.06 |
| SIS | 0.87 ± 0.07* |

*P < 0.01 significantly compared with control group

The data of Table 9-11 indicated that SIS could increase uterine and hormone function of female animal

### EXAMPLE 13

### Acute Toxicity Test

LD₅₀ (median lethal dosage) of SIS is 1050 mg/kg through abdominal injection in mice No differences between the animals of SIS group and normal animals were observed in symptoms and behavior.

Each dose for an adult is 20-50 kg. Using 50 kg as the average weight of an adult the dosage is 0.4-1 mg/kg Therefore it is very safe

The preparation of SIS is simple and can be accomplished by the extraction method set forth above or any conventional method for extracting the active ingredients from soybean residue The novelty of the present invention resides in SIS and in the preparation of dosage units in pharmaceutically acceptable dosage form The term “pharmaceutically acceptable dosage form” as used hereinabove includes any suitable vehicle for the administration of medications known in the pharmaceutical art, including, by way of example, tablets, capsules, syrups, elixirs, and solutions for parenteral injection with specified ranges of SIS concentration. The present invention provides novel method for increasing immune function, treating cardiovascular disease and decreasing lipids with easily produced, safe pharmaceutical agent.

It will thus be shown that there are provided compositions and methods which achieve the various objects of the invention, and which are well adapted to meet the conditions of practical use.

As various possible embodiments might be made of the above invention, and as various changes might be made in the embodiments set forth above, it is to be understood that all matters herein described are to be interpreted as illustrative and not in a limiting sense.

What is claimed as new and desired to be protected by Letter Patent is set forth in the appended claim:

1. A safe pharmaceutical composition for treatment and prevention of cardiovascular disease, menopausal and climacteric syndrome, and increasing of immunity using a soybean isoflavone extracted from residue and leaves of soybean

2. A safe pharmaceutical composition of claim 1 wherein the amount sufficient to treat and prevent of cardiovascular disease is about 20-50 mg of soybean isoflavone.

3. A safe pharmaceutical composition of claim 2, which is tablet or capsule

4. A dosage unit according to claim 2 wherein said dosage form is tablet including in addition pharmaceutical acceptable binder and excipients.

5. A dosage unit according to claim 2 wherein said dosage form is a solution for injection which includes in addition a liquid vehicle suitable for parenteral administration

6. A safe pharmaceutical composition of claim 1 wherein the amount sufficient to increase immunity is about 20-50 mg of soybean isoflavone

7. A safe pharmaceutical composition of claim 6, which is tablet or capsule

8. A dosage unit according to claim 6 wherein said dosage form is tablet including in addition pharmaceutical acceptable binder and excipients

9. A dosage unit according to claim 6 wherein said dosage form is a solution for injection which includes in addition a liquid vehicle suitable for parenteral administration.

10. A safe pharmaceutical composition of claim 1 wherein the amount sufficient to treat and prevent menopausal and climacteric syndrome is about 20-50 mg of soybean isoflavone.

11. A safe pharmaceutical composition of claim 10, which is tablet or capsule.

12. A dosage unit according to claim 10 wherein said dosage form is tablet including in addition pharmaceutical acceptable binder and excipients

13. A dosage unit according to claim 10 wherein said dosage form is a solution for injection which includes in addition a liquid vehicle suitable for parenteral administration

14. A process for producing soybean isoflavone for treatment and prevention of cardiovascular disease, menopausal and climacteric syndrome, and increasing of immunity from soybean residue comprising
(a) extracting a ground soybean residue with 95% ethanol at room temperature for 24 hours;
(b) filtering the above mixture and separating filtrate from residue,
(c) Ethanol was added to the filtrated residue and refluxed in water bath for 6 hours;
(d) the refluxing was repeated twice;
(e) the refluxed ethanol was cooled and filtered;
(f) the filtrate combined with the extract filtrate;
(g) ethanol was then recovered by reduced pressure distillation and residue dissolved in acetone,
(h) acetone was then recovered by reduced pressure distillation and residue was extracted by acetic ether,
(i) acetic ether was then recovered by reduced pressure distillation and the residue was vacuum dried, and
(j) the resulting white or light yellow powder was product.

15. A process for producing soybean isoflavone for treatment and prevention of cardiovascular disease, menopausal and climacteric syndrome, and increasing of immunity from soybean leaves comprising
(a) extracting a ground soybean residue with 95% ethanol at room temperature for 24 hours,
(b) filtering the above mixture and separating filtrate from residue,
(c) Ethanol was added to the filtrated residue and refluxed in water bath for 6 hours,
(d) the refluxing was repeated twice;
(e) the refluxed ethanol was cooled and filtered;
(f) the filtrate combined with the extract filtrate;
(g) ethanol was then recovered by reduced pressure distillation and residue dissolved in chloroform,
(h) chloroform was then recovered by reduced pressure distillation and residue extracted by butanol,
(i) butanol was recovered by reduced pressure distillation and residue was purified in column packed with sephadex and eluting with methanol,
(j) elution was extracted by acetone;
(k) acetic ether was then recovered by reduced pressure distillation and distillated residue was dried under vacuum, and
(l) the resulting white powder was soybean isoflavone
